(19) United States
(12) Patent Application Publication
(10) Pub. No.: US 2022/0133292 A1
(43) Pub. Date: May 5, 2022

(54) SURGICAL RETRACTOR

(71) Applicant: Spineology Inc., St. Paul, MN (US)

(72) Inventor: Todd Bjork, Hudson, WI (US)

(21) Appl. No.: 17/519,498

(22) Filed: Nov. 4, 2021

Related U.S. Application Data
(60) Provisional application No. 63/109,850, filed on Nov. 4, 2020.

(51) Int. Cl. A61B 17/02

(52) U.S. Cl. CPC A61B 17/0206; A61B 2017/0256; A61B 17/025

(57) ABSTRACT

A retractor can have a portal attached thereto. A portal attachment lock button allows for adjustment of the depth of the portal with respect to the patient. A portal alignment mechanism, such as a ball and socket, allows the portal to be maneuvered. A pair of lock releases control the opening and closing of a respective one of a pair of retractor blades independent from one another. A pair of toeing mechanisms can operate independently as well.

### PRIORITY

This application claims the benefit of U.S. Provisional Application Ser. No. 63/109,850, filed on Nov. 4, 2020, which is hereby incorporated herein by reference in its entirety.

### FIELD

The present invention generally relates to retractors. More particularly, the present invention relates to a retractor useful for creating a minimally invasive access opening to an intervertebral space.

### BACKGROUND

A variety of tools are available for surgeons for retracting tissue to create a surgical access. There is a continuing need to provide improved tools for retracting a patient's tissues.

### SUMMARY

The disclosure includes a retractor to which a portal can be attached. A portal attachment lock button allows for adjustment of the depth of the portal with respect to the patient. A portal alignment mechanism, such as a ball and socket, allows the portal to be maneuvered. A pair of lock releases control the opening and closing of a respective one of a pair of retractor blades independent from one another. A pair of toeing mechanisms can operate independently as well.

The detailed technology and preferred embodiments implemented for the subject invention are described in the following paragraphs accompanying the appended drawings for people skilled in this field to well appreciate the features of the claimed invention. It is understood that the features mentioned hereinbefore and those to be commented on hereinafter may be used not only in the specified combinations, but also in other combinations or in isolation, without departing from the scope of the present invention.

### BRIEF DESCRIPTION OF THE DRAWINGS

FIG. 1 is a top view of a surgical retractor in accordance with certain embodiments.

FIG. 2 is a perspective view of a surgical retractor in accordance with certain embodiments.

FIG. 3 is another perspective view of a surgical retractor in accordance with certain embodiments.

FIG. 4 is a side view of a surgical retractor in accordance with certain embodiments.

FIG. 5 is a top view of a surgical retractor in accordance with certain embodiments.

FIG. 6 is a front view of a surgical retractor in accordance with certain embodiments.

FIG. 7 is a front view of a surgical retractor in accordance with certain embodiments.

FIG. 8 is a top view of a surgical retractor in accordance with certain embodiments.

FIG. 9 is a perspective view of a surgical retractor in accordance with certain embodiments.

FIG. 10 is a top view of a surgical retractor in accordance with certain embodiments.

FIG. 11 is a front view of a surgical retractor in accordance with certain embodiments.

FIG. 12 is a side view of a surgical retractor in accordance with certain embodiments.

FIG. 13 is a perspective view of a surgical retractor in accordance with certain embodiments.

FIG. 14 is a top view of a surgical retractor in accordance with certain embodiments.

FIG. 15 is a front view of a surgical retractor in accordance with certain embodiments.

FIG. 16 is a top view of a surgical retractor in accordance with certain embodiments.

FIG. 17 is a front view of a surgical retractor in accordance with certain embodiments.

FIG. 18 is a side view of a surgical retractor in accordance with certain embodiments.

FIG. 19 is a top view of a surgical retractor in accordance with certain embodiments.

FIG. 20 is a front view of a surgical retractor in accordance with certain embodiments.

FIG. 21 is a side view of a surgical retractor in accordance with certain embodiments.

FIG. 22 is a perspective view of a surgical retractor in accordance with certain embodiments.

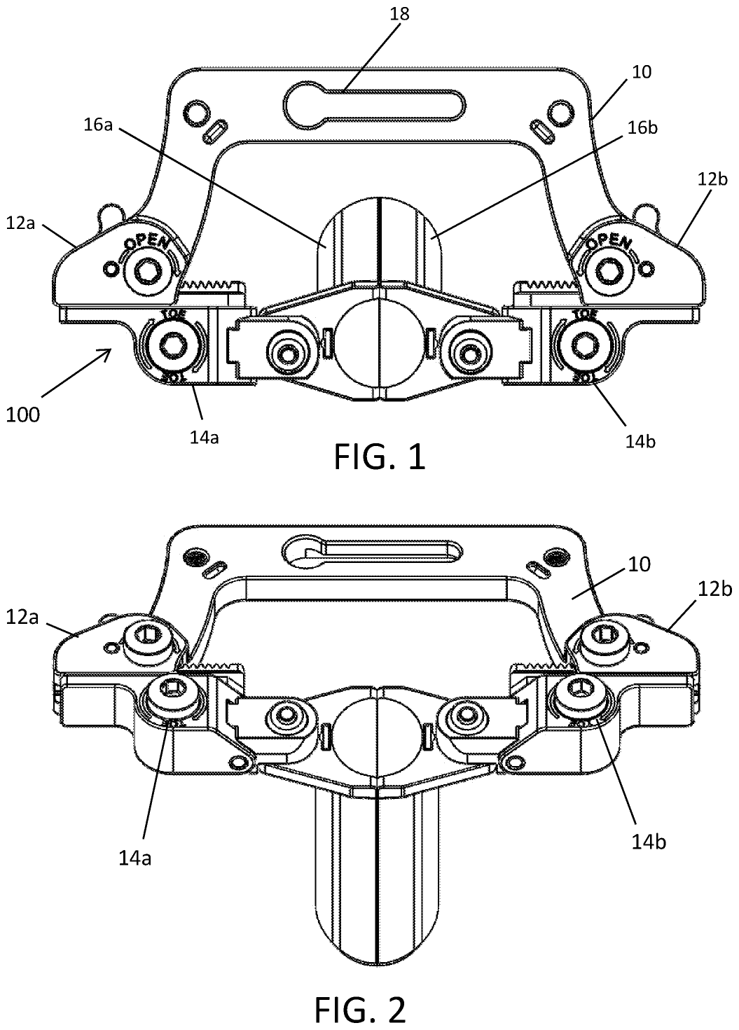

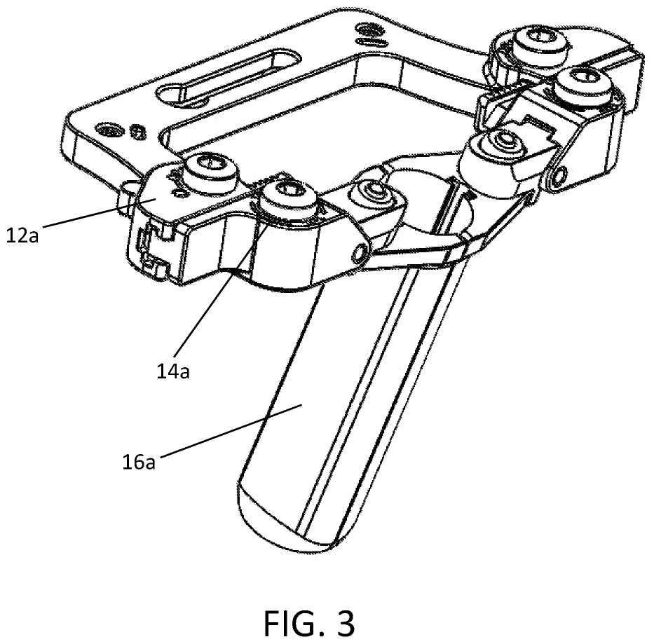

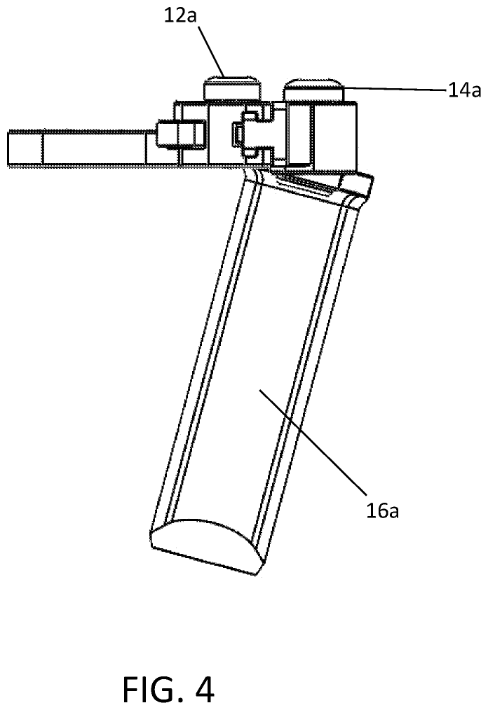

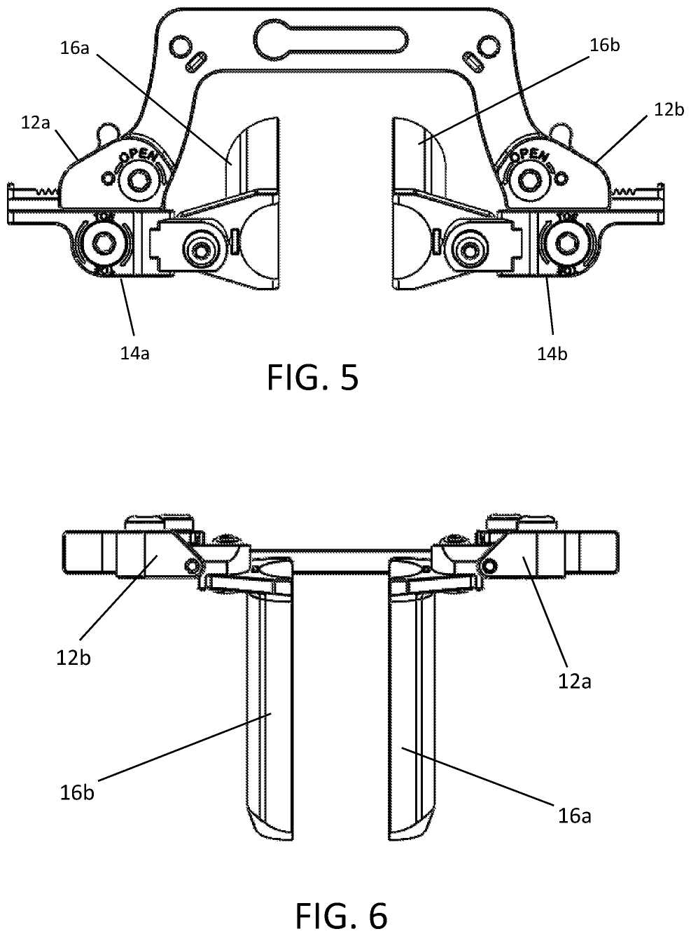

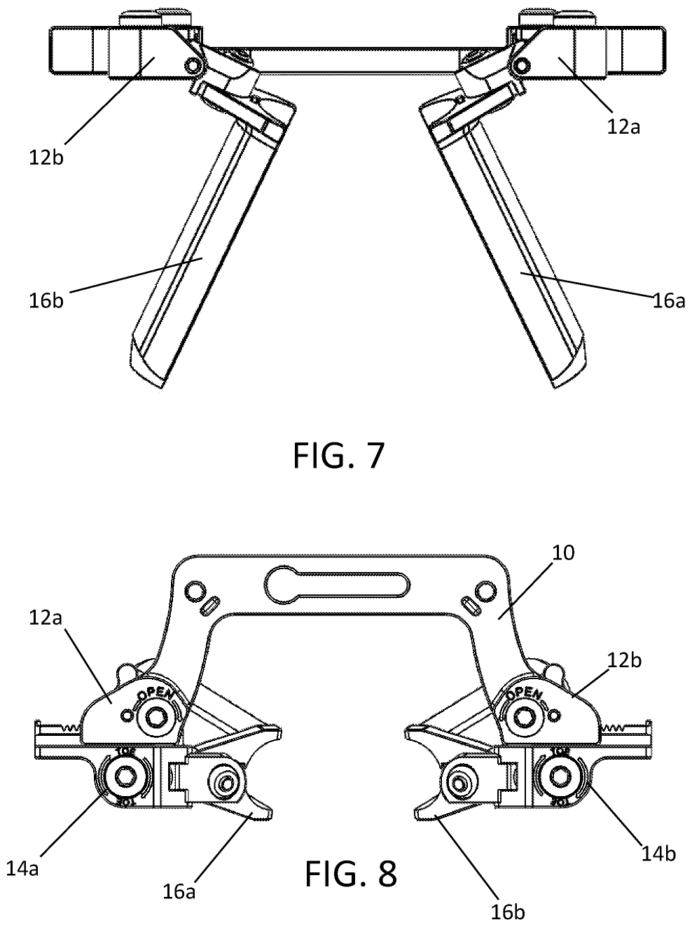

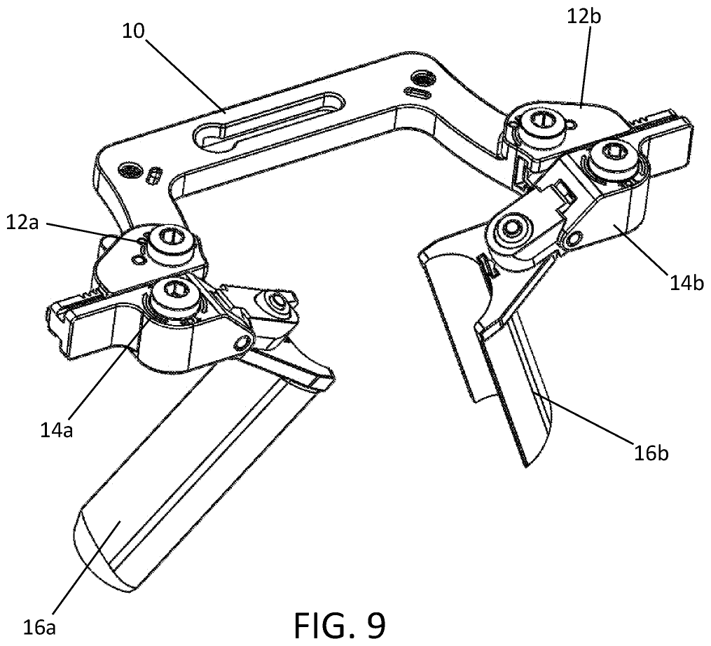

While the invention is amenable to various modifications and alternative forms, specifics thereof have been shown by way of example in the drawings and will be described in detail. It should be understood, however, that the intention is not to limit the invention to the particular example embodiments described. On the contrary, the invention is to cover all modifications, equivalents, and alternatives falling within the spirit and scope of the invention as defined by the appended claims.

### DETAILED DESCRIPTION

In the following descriptions, the present invention will be explained with reference to example embodiments thereof. However, these embodiments are not intended to limit the present invention to any specific example, embodiment, environment, applications or particular implementations described in these embodiments. Therefore, description of these embodiments is only for purpose of illustration rather than to limit the present invention. It should be appreciated that, in the following embodiments and the attached drawings, elements unrelated to the present invention are omitted from depiction; and dimensional relationships among individual elements in the attached drawings are illustrated only for ease of understanding, but not to limit the actual scale.

As can be seen throughout the figures, the surgical retractor 100 in certain embodiments includes a frame 10, lock releases 12a and 12b, toeing mechanisms 14a and 14b, retractor blades 16a and 16b, and a medial-lateral positioning guide 18. The retractor can also include a locking mechanism 20 for a portal attachment assembly, a portal attachment arm 22, a portal attachment lock button 24, and a portal alignment mechanism 26.

The lock releases 12a and 12b control the opening and closing of the blades 16a and 16b independent from one another. The toeing mechanisms 14a and 14b are independently operable as well.

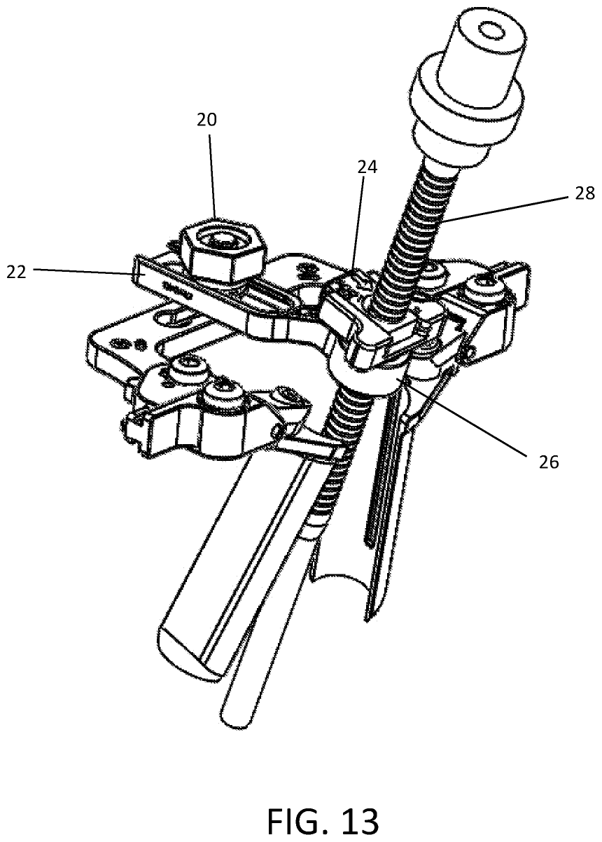

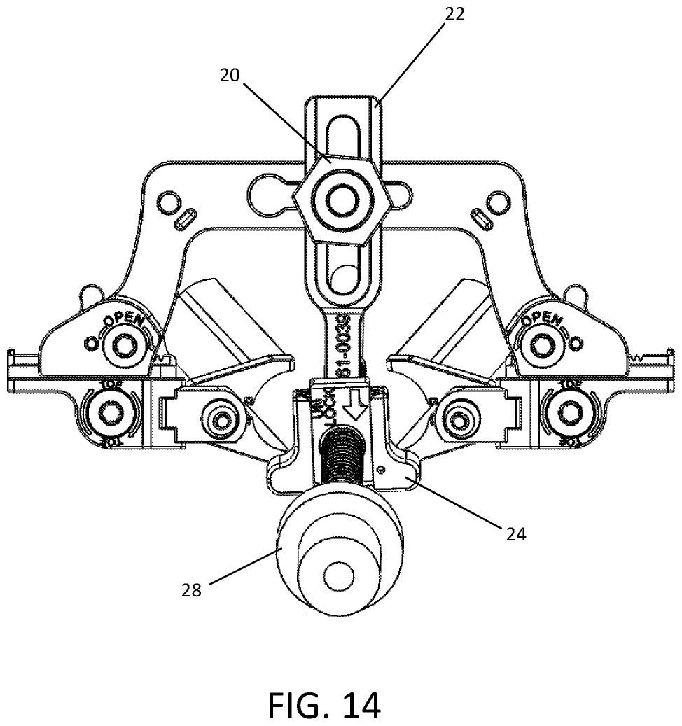

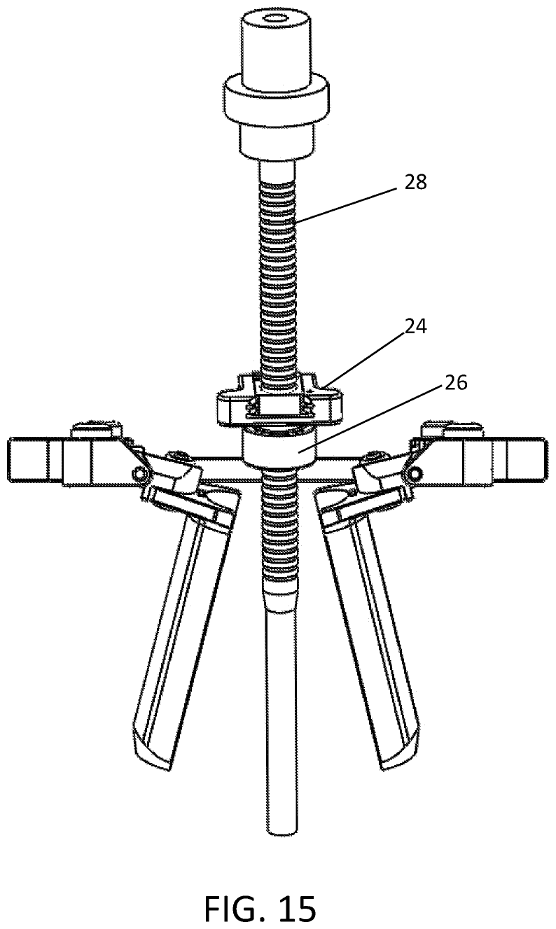

A portal 28 can be attached to the retractor 100 via the portal attachment arm 22. An example of this is shown in FIGS. 13-15. In such embodiment, the portal attachment lock button 24 provided to the portal attachment arm 22 allows for adjustment of the depth of the portal 28 with respect to the patient. The portal alignment mechanism 26 (in the figures a ball and socket) allows the portal 28 to be maneuvered by the user. The lock mechanism 20 is tightened to secure the legs of the portal attachment arm to the frame 10 at the user's desired location along the medial-lateral positioning guide 18.

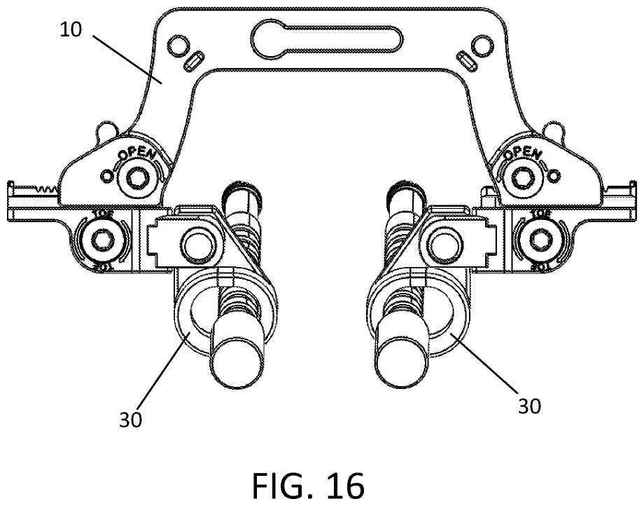

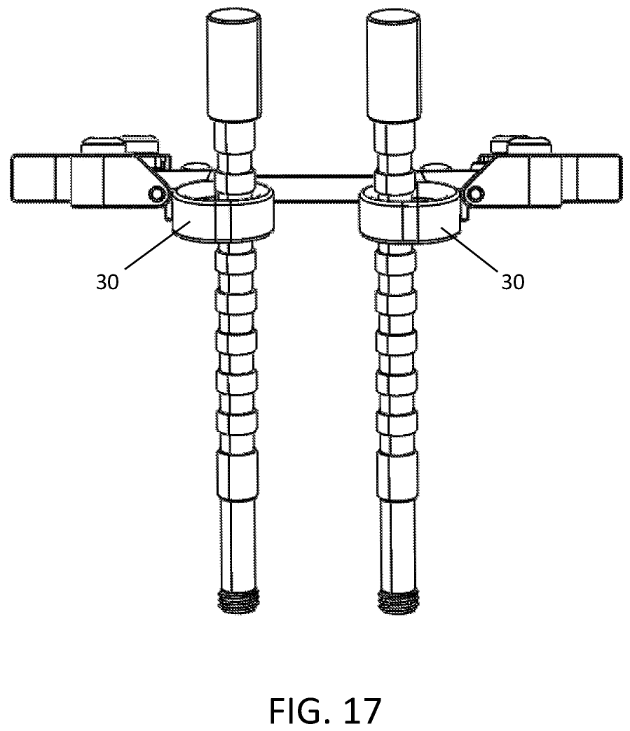

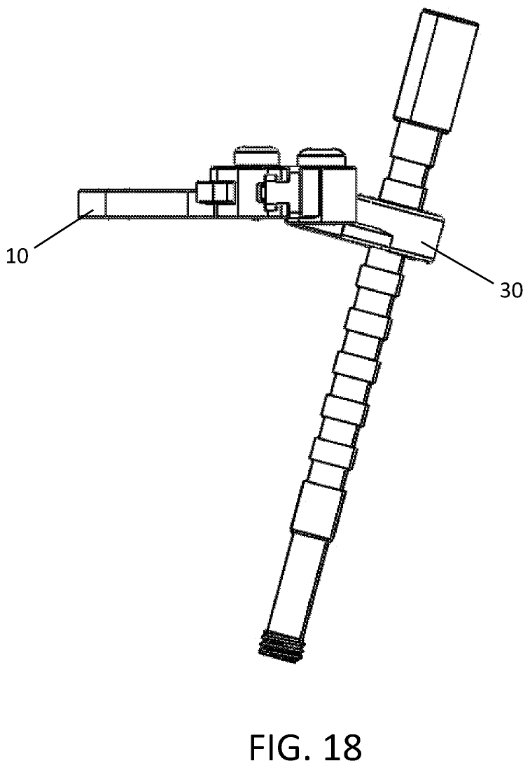

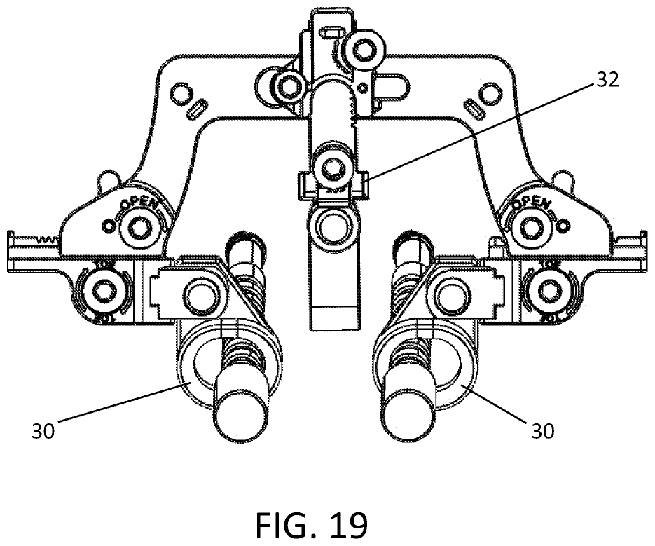

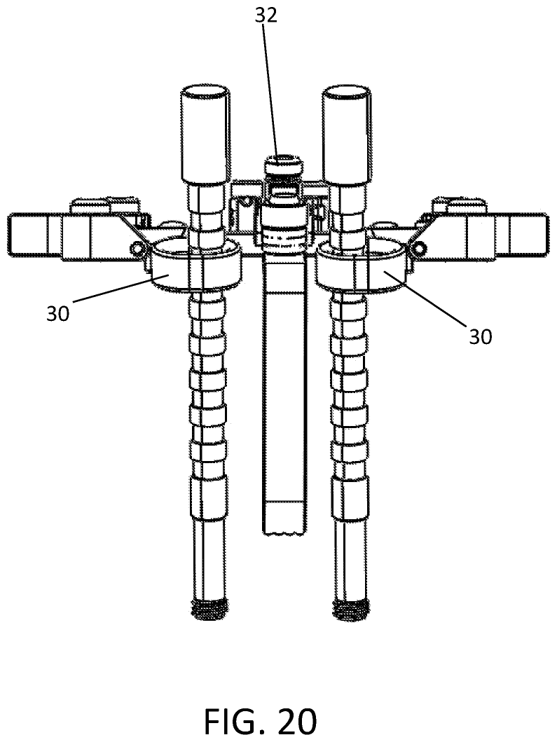

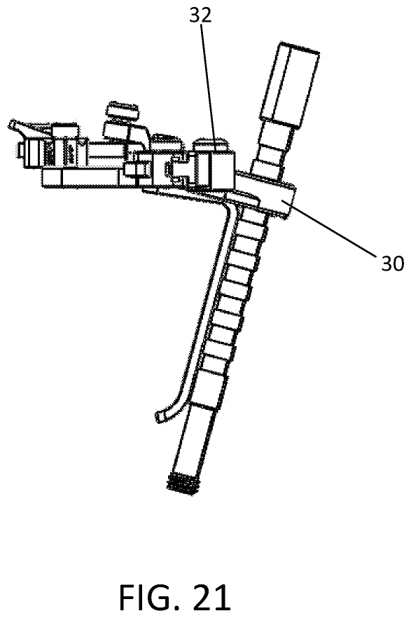

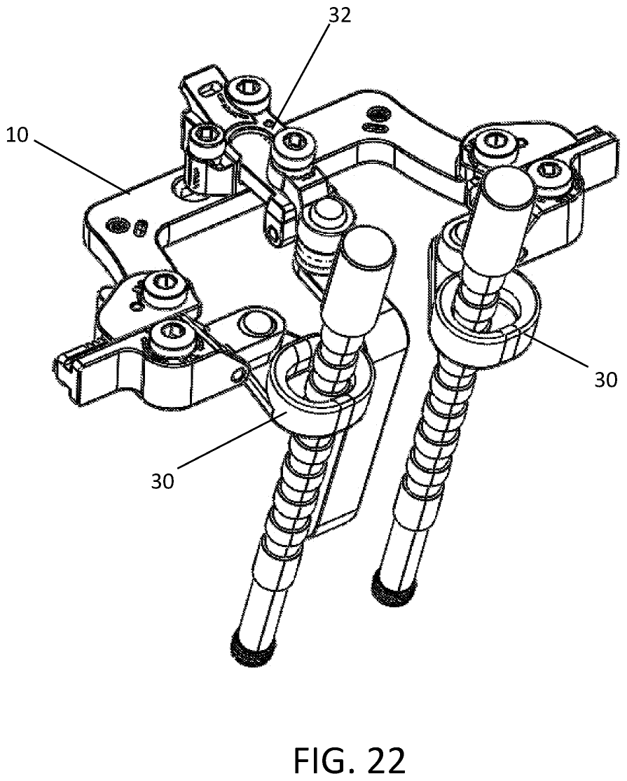

In a further embodiment, such as shown in FIGS. 16-22, a pair of pedicle screw hoop attachments members 30 can be provided to the frame 10 in place of the blades 16a and 16b.

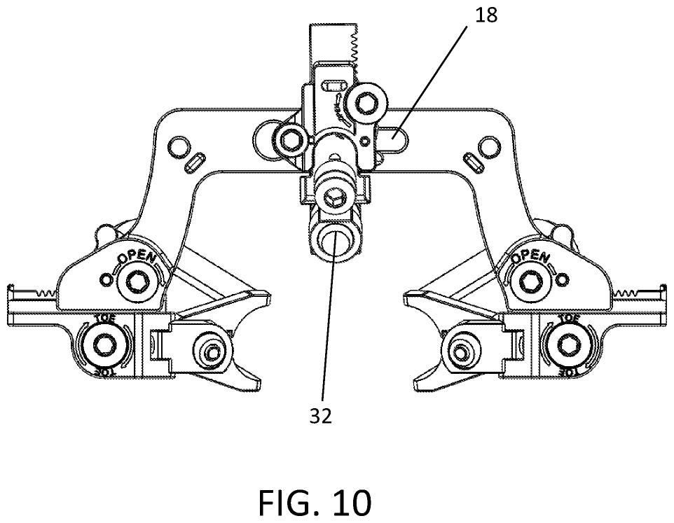

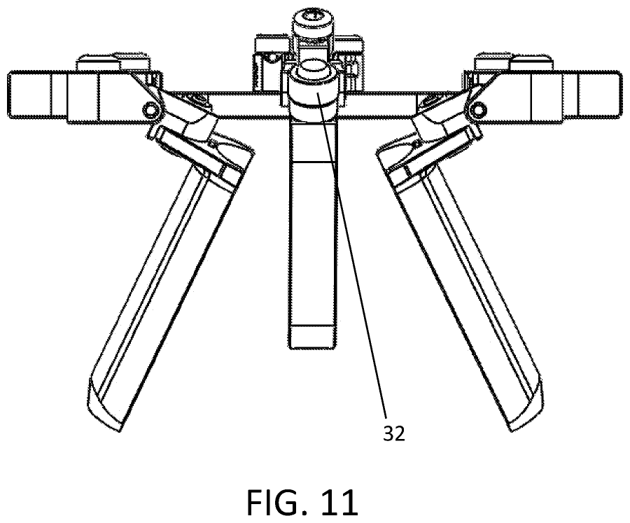

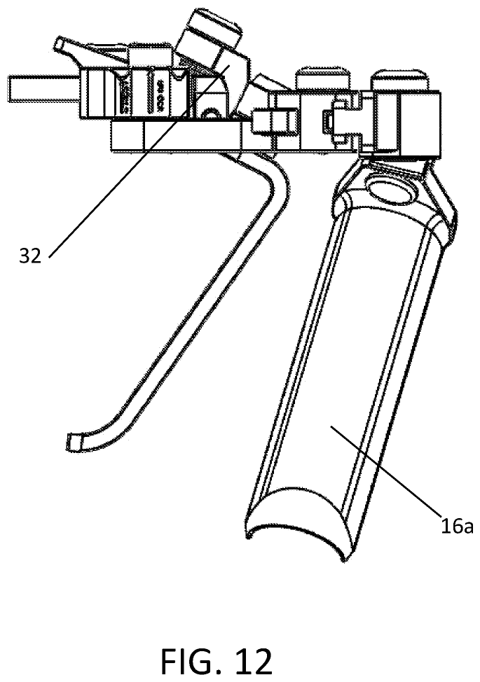

A medial blade assembly 32 can also be attached to the frame 10 as shown in FIGS. 10-12 and 19-22 along the medial-lateral positioning guide 18.

The entirety of U.S. Patent Application Pub. No. US 2019/0216450 A1 is hereby incorporated herein and made part of this application.

The present surgical retractor can be used in a surgical method or procedure such as a spinal fusion procedure, which is an additional aspect herein.

The present invention may be embodied in other specific forms without departing from the spirit or essential attributes thereof, and it is, therefore, desired that the present embodiment be considered in all respects as illustrative and not restrictive. Those skilled in the art may recognize other equivalents to the specific embodiment described herein which equivalents are intended to be encompassed by the claims attached hereto.

What is claimed is:

1. A surgical retractor, comprising:
a frame;
a pair of toe adjusters provided to the frame, wherein each of the tow adjusters are independently operable from one another;
a blade secured to each one of the toe adjusters;
a pair of lock releases provided to the frame, wherein each one of the lock releases is configured to control opening and closing of the blades and such control is independent for each lock release.

2. The surgical retractor of claim 1, further comprising a portal attachment arm secured to the frame.

3. The surgical retractor of claim 2, wherein the frame includes a medial-lateral positioning guide, wherein the surgical retractor further comprises a portal attachment arm, and wherein the portal attachment arm is secured to the frame via an adjustable lock.

4. The surgical retractor of claim 3, further comprising a portal attachment lock button provided to the portal attachment arm, the portal attachment lock button configured to adjust a portal depth dimension.

5. The surgical retractor of claim 3, further comprising a portal alignment mechanism that permits the portal to be maneuvered.

6. The surgical retractor of claim 5, wherein the portal alignment mechanism comprises a ball and a socket.
